(19) United States
(12) Patent Application Publication
(10) Pub. No.: US 2018/0055898 A1
(43) Pub. Date: Mar. 1, 2018

(54) TRADITIONAL CHINESE MEDICINE COMPOSITION FOR PREVENTING AND TREATING EXTERNALLY DIABETES AND A METHOD OF USING THE SAME

(71) Applicant: Tianfeng JIANG, ALHAMBRA, CA (US)

(72) Inventors: Tianfeng JIANG, ALHAMBRA, CA (US); Zhenghua JIANG, YANTAI (CN); Jinrong ZHOU, YANTAI (CN); Zhongyuan SUN, YANTAI (CN)

(21) Appl. No.: 15/683,625

(22) Filed: Aug. 22, 2017

(30) Foreign Application Priority Data
Aug. 29, 2016 (CN) 201610737865.5

(51) Int. Cl. A61K 36/481; A61K 36/537; A61K 36/536; A61K 36/605; A61K 36/284; A61K 36/8945; A61K 36/488; A61K 36/482; A61K 9/14

(52) U.S. Cl. CPC A61K 36/481; A61K 36/537; A61K 36/536; A61K 36/605; A61K 9/14; A61K 36/8945; A61K 36/488; A61K 36/482; A61K 36/284

(57) ABSTRACT

The invention discloses a traditional Chinese medicine composition and a method thereof for preventing and treating externally diabetes, which is characterized in that the traditional Chinese medicine composition is composed of astragalus membranaceus, salvia miltiorrhiza, stigma maydis, cortex lycii radicis, wolfberry, Prunella vulgaris, mulberry leaf, cornus officinalis, lycopodium clavatum, the root of bidentate achyranthes, rhizoma chuanxiong, Chinese atractylodes, yam, puerarin, fructus arctii, cassia twig; the method of use is to put a 40-50 g/ml medicine bags of the Chinese herbal medicine powder or decoction into a foot pot, brew with boiling water, and then steam more than 10 minutes with the traditional Chinese medicine steam after stirring evenly. When the water is cooled to 40-42° C., soaking feet once a day before going to bed, a course for foot soaking being 20 days; the contents of blood glucose and symptom of polydipsia, polyuria and polyphagia of the patients can be effectively reduced and disappeared by the foot bath in traditional Chinese medicine, thus improving the immunity of the body and activating the autoimmunity as well as promoting the self-healing ability for the disease; and also such diseases as diabetic peripheral neuropathy (DPN), diabetic foot, diabetic nephropathy (DN), uremia caused by renal insufficiency resulted from diabetes can be prevented.

### TECHNICAL FIELD

The present invention relates to the field of diabetes preventing and treating externally with traditional Chinese medicine, and more particularly, to a Chinese medicine composition for preventing and treating externally diabetes and a method of using the same.

### BACKGROUND

Diabetes is a chronic disease resulting in increased blood glucose and excretion of diabetes (urine contains glucose) caused by metabolic disorders of glucose, fat, protein, water and inorganic salt resulted from the reduction of insulin secretion in the body and genetic reasons. It affects mostly the middle-aged people. It is manifested clinically by “three to one less (sanduoyishao)”, namely, polydipsia, polyuria, polyphagia and weight reduction.

The direct cause of diabetes is the reduction of insulin secretion. If you are in depression and the spirit is stimulated, or have diseases such as concussion, encephalitis, cerebral hemorrhage, advanced neurological dysfunction would emerge and even the pituitary gland, adrenal gland, pancreas would be affected, thereby resulting in metabolic disorders due to neurohumoral regulation dysfunction. Additionally, increasing demand for insulin by the body due to all factors such as infection, pregnancy that can increase the metabolism leads to a relatively insufficient insulin secretion such that the glucose utilization in the body is affected to increase blood glucose. When the blood glucose concentration increases above the kidney threshold (that is the retention threshold of glucose in kidney; the glucose can be discharged from the urine usually when the blood glucose concentration is above 160-180 mg %), the glucos-uria appears to be diabetes.

Diabetic patients are often prone to complications such as suppurative infection, tuberculosis, atherosclerosis, nerves, kidney, eye lesions, which may endanger life when ketoacidosis occurs severely.

Early diabetes often has no obvious symptoms, often found by the doctor in the event of complications. Diabetes is generally characterized by the symptoms of weakness, dizziness, drowsiness, backache leg pain, dry skin or itching, men impotence, women's irregular menstruation.

Diabetes is medically within “emaciation-thirst diseases (xiaoke)”. Chinese traditional medicine thinks that this disease is mostly caused by lung, stomach, kidney dryness due to yin deficiency leading to diabetes (xiaoke) resulted from alcohol and strong taste addiction, damage of spleen and stomach, movement and transformation failure, internal heat, swift digestion and fluid consumption in stomach; or indulgence to damage the yin, hepatic stagnation to fire.

There are areas in human feet, i.e. the reflective area, which corresponds objectively to various organs in the body. The physiological function of the body's organs can be regulated by stimulating these reflective areas, thereby achieving the purpose of treating diseases and self-care. Our feet have 62 basic transmit areas, which are in whole similar to a microcosm of the human body that concerns about the health status of each parts in human body all the time. Medicine absorption and delivery of medicine can be improved and accelerated by stimulating the reflective areas regularly with herbs feet bathing to facilitate smoothness of blood, function regulation of each organ under the dual role of stimulating induction in reflective areas and the medicine, thereby improving the pathological changes for organs and the body's immunity.

With the improvement of life quality, people pay more and more attention to health care; they soak feet in hot water every day to promote blood circulation and eliminate fatigue; those who focus more on health care soaking their feet with traditional Chinese medicine. The latter utilized the principle that the boiling water can foster the medicine to filtrate into the human body, so as to ensure the medicine to penetrate the whole body meridian through the foot and adverse reactions resulted from oral overdose not to occur.

Diabetes is a common metabolic endocrine disease, and is listed as one of the top ten difficult diseases by the World Health Organization. The incidence of this disease is extremely high, and tends to increase year by year; according to New York Times, a recent study shows that the prevalence of diabetes in China ranks first in the world, and about one-third of the world's diabetes patients are in China It is even worried that the blood glucose of a half has reached the “pre-diabetes” level, a phenomenon which is found after testing on nearly 9.9 thousand people by this study. “China is currently one of the countries with the highest prevalence of diabetes in Asia and the country with the largest number of people with diabetes in the world,” the study said. Now, there are more than 92.4 million people with diabetes, and one person is suffering from diabetes every 30 seconds. And also this situation continues to deteriorate, so the prevention and treatment for diabetes is a top priority, and is the most important.

At present, the treatment for diabetes mainly uses western medicine and insulin, and patients need to take drugs and fight insulin throughout their life; western medicine and insulin have large side effects, monotonous curative effects, and mostly aims at solving the problem of blood glucose increasing temporarily while the symptom is cured but the roots have not; in recent years, the theoretical study on diabetes and clinical efficacy have greater progress than ever before. But as western medicines still have greater effects on the damage of metabolism and organs and most have some side effects, having medicines for a long time not only hurts the body, but also is easy to allow the virus, bacteria to produce resistance, thus limiting its clinical use and making the treatment control be undesired.

It has a long history for treating diabetes by traditional Chinese medicine. However, daily decoction of traditional Chinese medicine costs time and efforts with complicated procedures. In general, the traditional Chinese medicine is administrated orally with a larger dose and has a poor taste. Long-term administration of traditional Chinese medicine also poses certain side effects on human body to hurt the body and be apt to allow the virus and bacteria to produce resistance such that the treatment effect is less and less obvious.

### SUMMARY

The purpose of the present invention is to overcome the above-described deficiencies of the prior art by providing a traditional Chinese medicine composition for the prevention and treatment of diabetes.

Another purpose of the present invention is to provide a method for using the Chinese medicine composition to prevent and externally treat diabetes.

The present invention mainly solves problems of the treatment for diabetes mainly using western medicine and insulin that have large side effects, monotonous curative effects, and mostly aims at solving the problem of blood glucose increasing temporarily with no consideration of comprehensive treatment; using traditional Chinese medicine that has side effects, time and energy cost for daily decoction and poor taste.

The technical proposal of the present invention comprises a traditional Chinese medicine composition for preventing and treating externally diabetes, characterized in that the traditional Chinese medicine composition is composed of Astragalus membranaceus, salvia miltiorrhiza, stigma maydis, cortex lycii radicis, wolfberry, Prunella vulgaris, mulberry leaf, cornus officinalis, lycopodium clavatum, the root of bidentate achyranthes, rhizoma chuanxiong, Chinese atractylodes, yam, puerarin, fructus arctii, cassia twig.

A traditional Chinese medicine composition for preventing and treating diabetes according to the present invention is composed of raw materials with the following weight ratio: 40-50 parts of astragalus membranaceus, 35-40 parts of salvia miltiorrhiza, 50-60 parts of stigma maydis, 40-50 parts of cortex lycii radicis, 25-30 parts of wolfberry, 25-30 parts of Prunella vulgaris, 25-30 parts of mulberry leaf, 25-30 parts of cornus officinalis, 35-40 parts of lycopodium clavatum, 30-35 parts of the root of bidentate achyranthes, 40-50 parts of rhizoma chuanxiong, 25-30 parts of Chinese atractylodes, 25-30 parts of yam, 25-30 parts of puerarin, 25-30 parts of fructus arctii, 25-30 parts of cassia twig.

A traditional Chinese medicine composition for preventing and treating diabetes according to the present invention comprises a better weight ratio: 35-45 parts of astragalus membranaceus, 32-38 parts of salvia miltiorrhiza, 40-50 parts of stigma maydis, 35-45 parts of cortex lycii radicis, 26-29 parts of wolfberry, 26-29 parts of Prunella vulgaris, 26-29 parts of mulberry leaf, 26-29 parts of cornus officinalis, 36-39 parts of lycopodium clavatum, 31-34 parts of the root of bidentate achyranthes, 42-48 parts of rhizoma chuanxiong, 26-29 parts of Chinese atractylodes, 26-29 parts of yam, 26-29 parts of puerarin, 26-29 parts of fructus arctii, 25-30 parts of cassia twig.

A traditional Chinese medicine composition for preventing and treating externally diabetes according to the present invention, the optimum weight ratio is: 45 parts of astragalus membranaceus, 38 parts of salvia miltiorrhiza, 55 parts of stigma maydis, 45 parts of cortex lycii radicis, 28 parts of wolfberry, 28 parts of Prunella vulgaris, 28 parts of mulberry leaf, 28 parts of cornus officinalis, 38 parts of lycopodium clavatum, 33 parts of the root of bidentate achyranthes, 45 parts of rhizoma chuanxiong, 28 parts of Chinese atractylodes, 28 parts of yam, 28 parts of puerarin, 28 parts of fructus arctii, 28 parts of cassia twig.

Further, the traditional Chinese medicine composition is a powder or a decoction.

A method for using the traditional Chinese medicine composition to prevent and treat externally diabetes according to the present invention is characterized in that it comprises the following steps: putting a 40-50 g/ml medicine bag of Chinese herbal medicine powder or decoction into a foot pot, brewing with 2000-3000 ml of boiling water, and then steaming more than 10 minutes with the traditional Chinese medicine steam after stirring evenly; when the water is cooled to 40-42° C., soaking your feet for 30-40 minutes; soaking feet once a day before going to bed and one course for foot bath is 20 days.

A traditional Chinese medicine composition for preventing and treating externally diabetes according to the present invention, the functions of the raw materials being as follows:

Astragalus membranaceus: sweet taste, humid; into lung, spleen meridian; having efficacy of benefiting Qi and raising Yang, consolidating exterior for arresting sweating, inducing diuresis for removing edema, generating saliva and nourishing blood, smoothing and freeing the meridian, expelling pus and drawing toxin, astringing sores and promoting granulation; used for those who has spleen qi deficiency, middle energizer dysfunction, sinking of middle qi, organ prolapse, lung qi deficiency, cough with dyspnea, exterior deficiency and spontaneous sweating, yin deficiency and night sweat, qi deficiency and water retention, oliguria and edema, qi and blood deficiency, deficiency of booth qi and the blood, failure to control blood, qi-deficiency and blood impediment, blood stasis, fluid consumption, qi and blood deficiency etc.; in modern study, have pharmacological effects on immune system, body metabolism, cardiovascular system, aging-resistance, urinary system, liver protection, nerve and endocrine system; clinically used for preventing and treating upper respiratory tract infection, pediatric asthma, plateau pulmonary heart disease, for treating viral myocarditis, heart failure, ischemic heart disease, chronic nephritis, rhinitis, myocardial infarction, cerebral thrombosis and viral myocarditis etc.

Salvia miltiorrhiza: bitter, slightly cold, into heart, liver meridian; having efficacy of activating blood and regulating menstruation, dispelling stasis and relieving pain, cooling blood and resolving carbuncle, clearing heart and eliminating oppression; used for menstrual irregularities, postpartum body pain, gastric abscess, warm febrile disease, rubella, skin itch, irritable feverish sensation in the chest, palpitation and severe palpitation, insomnia and amnesia etc.; in modern study, having effects on cardiovascular system, myocardial ischemia, myocardial infarction, blood system, cardiac hypertrophy, liver, promotion for tissue regeneration, tumor, central nervous system and clinical effects on blood lipids and atherosclerotic plaque formation; clinically used for treating cerebrovascular disease, coronary heart disease, pulmonary heart disease, pediatric viral myocarditis, acute and chronic hepatitis, acute and chronic nephritis, numbness of the lower limbs, epidemic hemorrhagic fever, diabetes etc.

Stigma maydis: sweet, mild, into liver, kidney, bladder meridian; having efficacy of inducing diuresis for removing edema, dispersing stagnated liver qi to promote bile flow; used to treat edema and stranguria, liver yang headache, yang jaundice and yin jaundice etc.; in modern study, having effects on hypolipidemic, hypoglycemic, anti-oxidation, immune system, anti-bacteria; clinically used to treat chronic glomerulonephritis, urinary calculus, type 2 diabetes mellitus (T2DM), icterohepatitis, chronic biliary infection and newly diagnosed hypertension etc.

Cortex lycii radicis: sweet, cold; into lung, liver, kidney meridian; having efficacy of cooling blood and removing steaming, clearing lung fire; used for yin-deficiency fever, night sweat and steaming bone fever, lung heat and cough, hemorrhagic fever, internal heat and consumptive thirst etc.; in modern study, having effects on antipyretic, hypoglycemic, analgesic, antimicrobial, immunomodulatory, anti-fertility etc.; clinically used to treat diabetes, essential hypertension, menopause metrorrhagia and metrostaxis, pulpitis, lymphoid tuberculosis, allergic dermatosis etc.

Wolfberry: sweet, mild; into liver, spleen meridian; having efficacy of nourishing liver and kidney, replenishing vital essence to improve eyesight; used for kidney deficiency and atrophic debility of bones, impotence and seminal emission, premature senescence, early white of beard and hair, blood deficiency and chlorosis, fatigue damage to weakness, unclear eyes, internal heat and consumptive thirst, wind stroke (apoplexy) to dizzy, dysphoria to insomnia, non-productive cough with little sputum etc.; in modern study, having effects on body immune system, anti-tumor, anti-oxidation, anti-aging etc.; clinically used to treat infertility, delay aging, hyperemesis gravidarum, diabetes, hyperlipemia etc.

Prunella vulgaris: bitter, spicy, cold, non-toxic; into liver, gallbladder meridian; having efficacy of removing liver fire for improving eyesight, cooling blood to stop bleeding, reducing swelling and resolving mass, cough relieving and sputum elimination, Heat-clearing and detoxicating; used for lymphoid tuberculosis, acute mastitis, goiter, massive vaginal bleeding, dizziness, tuberculosis, distortion of the face, sinews and bones pain, leukorrhea, acute infectious icterichepatitis and bacillary dysentery etc.; in modern study, having effects on antihypertensive, anti-inflammation, anti-bacterial, inhibit immunity, hypoglycemic etc.

Mulberry leaf: bitter, sweet, cold; into lung, liver meridian; having efficacy of dissipating wind-heat (pityriasis), clearing lung-heat and moistening drynes, removing liver fire for improving eyesight; used for wind-heat type common cold, incpiency of warm disease, lung heat cough, scarlatina, garget, toothache, red eye swelling and pain, wind tears, liver yang dizziness, giddiness, hemorrhage, spontaneous sweating, night sweating, rubella etc.; in modern study, having effects on anti-inflammation, hypoglycemic, antilipemics, anti-oxidation, anti-aging, anti-bacteria etc.; clinically used to treat cough, lung abscess, night sweating, apoplexy, dermatosis etc.

Cornus officinalis: sour, mild, into liver, kidney meridian; having efficacy of nourishing liver and kidney, controlling nocturnal emission and reducing urination, consolidating channel for hemostasis, relieving excessive sweat and preventing from sweating; used for soreness and weakness of waist and knees, dizziness and tinnitus, impotence, spermatorrhea, metrorrhagia, profuse sweating etc.; in modern study, having effects on immunomodulatory, hypoglycemic, antiarrhythmic, anti-oxidation, anti-bacteria etc.; clinically used to treat chronic primary thrombocytopenic purpura, salty taste in mouth, frozen shoulder, alopecia, hyperlipidemia, dizziness etc.

Herba lycopodii: bitter, spicy, mild, into liver, spleen, kidney meridian; having efficacy of expelling evil-wind and remove dampness, stimulating the circulation of the blood and causing the muscles and joints to relax; used to treat wind-cold and warm arthralgia; in modern study, having effects of anti-inflammation, analgesia; clinically used to treat achillobursitis and diabetic peripheral neuropathy etc.

The root of bidentate achyranthes: bitter, sweet, sour, mild; into liver, kidney meridian; having efficacy of activating blood flow, conducting the heat downward, nourishing liver and kidney, strengthening sinews and bones, increasing-urine and removing-stone; used for dysmenorrheal and amenorrhea, traumatic injuries, upper-heat syndrome, soreness and weakness of waist and knees etc.; in moder study, having effects on immunomodulatory, anti-aging, anti-tumor, cardiovascular system, anti-inflammation and analgesia, diuresis; clinically used to expand cervical canal, treat dysfunctional uterine bleeding, pediatric pneumonia, arthritis, postoperative intestinal adhesion etc.

Rhizome chuangxiong: spicy, mild; into liver, gladder, heart meridian; having efficacy of activating blood and moving qi, expelling wind and alleviating pain, moving Qi and removing Qi-stagnation, used for lateral thorax pain, chest stuffiness and pains, traumatic injuries, ulcer and sore pains, menstrual irregularities, dysmenorrheal and amenorrhea, pantalgia after childbirth, headache, toothache, wind-dampness and arthralgia and red eye swelling etc.; in modern study, having pharmacological effects on cardiovascular system, coronary flow, brain circulation, microcirculation, peripheral blood vessels and blood pressure, blood system, smooth muscle, urinary system, respiratory system, central nervous system, anti-radiation; clinically used to treat coronary heart disease, ischemic brain disease, craniocerebral trauma, prevent and treat migraine, treat severe pulmonary heart disease, bronchial asthma, severe hepatitis, chronic nephritis and pregnancy-induced hypertension etc.

Chinese atractylodes: spicy, bitter, mild; into spleen, stomach, liver meridian; having efficacy of drying dampness and invigorating spleen, dispelling wind and expelling coldness, improving vision; used for dampness obstruction of middle energizer, dysfunction of spleen in transportation, wind cold dampness, wind cold dampness arthralgia, cardiodynia, dysentery pain etc.; in modern study, having effects on digest system, anti-ulcer, gastric emptying promotion, liver protection, anti-inflammation, hypoglycemic, antiarrhythmic; clinically used to treat autumn diarrhea, infantile anorexia, gastroptosis, diabetes, eczema etc.

Common yam rhizome: sweet, mild; into spleen, lung, kidney meridian; having efficacy of tonifying spleen and stomach, regenerating body fluid and benefiting lung, tonifying Kidney to control nocturnal emission; used for deficiency of spleen and stomach, cough due to lung deficiency, spermatorrhea due to kidney deficiency, slightly frequent leukorrhea, internal heat to diabetes etc.; in modern study, having effects on digest system, immune function regulation, hypoglycemic, hypolipidemic, anti-oxidation and anti-aging etc.; clinically used to treat diarrhea, diabetes, chronic nephritis, benign prostatic hyperplasia etc.

Puerarin: spicy, sweet, cold; into spleen, stomach, lung meridian; having efficacy of releasing flesh and removing heat, promoting fluid production to quench thirst, promoting eruption, elevating yang for anti-diarrhea, dredging channels and activating col laterals, antialcoholism; used for wind-cold, wind-heat, disability to promote eruption, constipation, spleen-deficiency diarrhea etc.; in modern study, having effects on smooth muscle, heart and brain, blood pressure, hypoglycemic, hypolipidemic, anti-tumor; clinically used to treat sciatica, cervical spondylosis, ischemic infarction, headache, torticollis, coronary heart disease etc.

Fructus arctii: spicy, bitter, cold; into lung, gastric meridian; having efficacy of dispelling wind-heat, releasing lung to promote eruption, removing toxin and relieving sore throat; used for wind-heat type common cold, warm disease incipiency, measles with no promotion, pruritus due to rubella, furuncle swelling and sore toxin, scrofula and phlegm node, pestilential toxin and suppurative parotitis, mumps and pharyngitis, sore throat, cough and asthma with much sputum etc.; in modern study, having effects on anti-virus, relieving cough, hypoglycemic, anti-nephritis, anti-oxidation, anti-tumor; clinically used to treat facial paralysis, nasosinusitis, acute mastitis, arthritis and diabetic nephropathy etc.

Cassia twig: spicy, sweet, mild, into heat, lung, gladder meridian; having efficacy of sweeting and releasing flesh, warming meridian/channel, assisting yang transformation to qi, descending adverse-rising qi; used for wind-cold type common cold, palpitation and chest impediment, epigastric pain due to cold, amenorrhea and abdominal mass, wind-warm impediment, drink stoppage due to cold-phlegm, cough and edema, palpitation and vomit, liver stagnation and wind, cold fever due to malaria, profuse sweating and seminal emission, hemorrhagic fever etc.; in modern study, having effects on relieving heat, relieving pain, producing sweat, anti-bacteria, anti-inflammation, improving blood circulation, hypotensive, anti-tumor, promotion of bones etc.; clinically used to treat dysmenorrheal, menstrual migraine, hysteromyoma, chronic asthma, cardiovascular disease etc.

The traditional Chinese medicine composition composed of above traditional Chinese medicines, according to the principle of dialectical treatment of traditional Chinese medicine, can have effects on nourishing yin, regenerating body fluid, nourishing stomach, clearing lung, reinforcing the vital energy and consolidating the constitution, replenishing and benefiting kidney, lung and spleen; and in comprehensive way, such regulations on body balance as antihypertensive, hyperglycemic, lipidlowering; mainly on improving the immunity of the body and strengthen the metabolism, thereby inhibiting, regulating to lower blood glucose level and to eliminate symptom of polydipsia, polyuria, polyphagia (sanduo). In general, after one course of foot bath with the medicine, the symptom is felt to disappear by oneself. It also has efficacy of lowering blood glucose level, enforcing spleen and consolidating kidney, harmonizing the five internal organs (heart, liver, spleen, lungs and kidneys), activating blood and dredging collaterals and dispelling wind and relieving pain; it prevents and treats such phenomenon as diabetic peripheral neuropathy, diabetic foot, diabetic nephropathy and harmfulness on human life from uremia caused by insufficient function of kidney due to diabetes. It has a significant effect on preventing and treating headache, feebleness, numbness of hands and feet and complications resulted from diabetes. At the same time, occurrence and development of the complications can be controlled and prevented to reach the purpose of curing diabetes completely.

A traditional Chinese medicine composition for preventing and treating externally diabetes according to the present invention, the clinical experiment data of which are as follows:

Process: weighing accurately 40 g astragalus membranaceus, 38 g salvia miltiorrhiza, 55 g stigma maydis, 45 g cortex lycii radicis, 28 g wolfberry, 28 g prunella vulgaris, 28 g mulberry leaf, 28 g cornus officinalis, 38 g lycopodium clavatum, 33 g the root of bidentate achyranthes, 45 g rhizoma chuanxiong, 28 g Chinese atractylodes, 28 g yam, 28 g puerarin, 28 g fructus arctii, 28 g cassia twig; grinding the mixture of above traditional Chinese medicines into 160-200 mesh sieve powder, then putting them into several small bags made of nonwovens respectively, each being 45 g, and enclosing as backups.

Application method: putting a 40 g medicine bag into a foot pot, brewing with 2000-3000 ml of boiling water, and then steaming more than 10 minutes with the traditional Chinese medicine steam after stirring evenly; when the water is cooled to 40-42° C., soaking your feet for 30-40 minutes; soaking feet once a day before going to bed; a course for foot soaking is 20 days.

Data manifestation: 60 samples including half male patients and half female patients who are 40-80 years old, wherein type 1 diabetic patient accounts for 50% and type 2 accounts for 50%.

Observation index for efficacy: observing the improvement of blood glucose and “polydipsia, polyuria, polyphagia and weight reduction (three to one less)” of a diabetic patient, and stability and recurrence after blood glucose level back to normal.

Evaluation for therapeutic effect:

Having a foot bath every day before going to bed, then the symptom of “polydipsia, polyuria, polyphagia (sanduo)” is felt to disappear, wherein fasting plasma glucose (FPG)≦7.8 mmol/L; but has not decreased to normal level. This refers to “effective”.

Having a foot bath everyday with the traditional Chinese medicine composition of the present invention and stopping administration after two courses, then the symptom of “polydipsia, polyuria, polyphagia (sanduo)” is felt to be improved and the FPG decreases to normal level. The blood glucose is being normal in six months after administration. This refers to “markedly effective”.

Having a foot bath once every day, then the blood glucose is slightly lower than original after one course and decreases after 3 courses but still higher than normal level, and the blood glucose is unstable, the symptom of “polydipsia, polyuria, polyphagia (sanduo)” has no improvement but even gets worse. This refers to “ineffective”.

Summary: In above 60 samples, 54 are “effective”, accounting for 90%; 40 are “markedly effective”, accounting for 67%; 6 are “ineffective”, accounting for 10%.

Typical cases of clinical experiments are as below:

1. Mrs. Liu, 80 years old, was diagnosed to have diabetes 20 years ago, FPG being 6.3 (mmol/L). At that time, she felt nothing and didn't have medicine; but after several years, she felt weak, backache and thirst. So when she went to the hospital, FPG was about 8.9 (mmol/L). The doctor then suggested her to take medicine incessantly. Then after taking medicine for a few years, the blood glucose had been always unstable. Five years ago, her FPG was still above 7.8 (mmol/L) while taking medicines. Finally, she began insulin injection, which is complicated and has side effects on body. However, after she had foot bath before going to bed every night with the traditional Chinese medicine of the present invention, once for 30-40 mins And at the same time, she was taking insulin injection. After one course, the blood glucose decreased and she felt vitalized; symptom of “polydipsia, polyuria, polyphagia” is felt to disappear. And after three courses of continuing to do that, FPG was 6.0 (mmol/L), the skin became dry, the itching symptom disappeared. Then after she stopped injecting insulin and continued to have foot bath before going to bed every day with the traditional Chinese medicine of the present invention for 6 months, she was re-examined to have 6.2 (mmol/L) FPG, which is normal. She hasn't taken any medicine since then.

2. Mrs. Wang, 48 years old, was examined in 2014 to find that the blood glucose was slight higher and was still high when being re-examined Her FPG was 6.5 (mmol/L), which belongs to type 1 diabetes. The doctor suggests her to take medicine for a long time without stoppage. With consideration to her age and body situation, the patient was subjected to conservative treatment for an attempt. She was introduced to have foot bath before going to bed every day with the traditional Chinese medicine of the present invention for 30-40 mins once a time while taking no medicines. After one course, the blood glucose became normal but unstable with occasional high level. After two courses with continuing to have foot bath before going to bed every day with the traditional Chinese medicine of the present invention, the FPG was about 5.8 (mmol/L) and she felt that symptom of “polydipsia, polyuria, polyphagia” had disappeared while body was becoming normal. So she began to stop taking medicine. Then continuing to do that allows her blood glucose to remain normal until now without relapse and taking medicine.

3. Mr. Zhao, 70 years old, having had diabetes for more than 10 years, having taken medicine for a long time, was a type 2 diabetes patient. His blood glucose level was up to 8.5 (mmol/L). He felt sleepy every day, weak, dizzy drowsiness and had a severe symptom of skin itching. The under the instruction of doctor, he began to insist on medication. When the disease is serious, he would take more, and when the blood glucose decreased, he would take normal dosage. He had foot bath before going to bed every day with the traditional Chinese medicine of the present invention for 30-40 mins once a time while taking medicines as usual. Then after 1 course, the blood glucose decreased a little but was still high. After 3 courses with continuing to have foot bath before going to bed every day with the traditional Chinese medicine of the present invention, the FPG decreased to about 6.5 (mmol/L). And continuing to have foot bath before going to bed everyday with the traditional Chinese medicine of the present invention until now while taking medicine as usual, the blood glucose became normal without taking any more medicines. Also, he became strong, the symptom of sleepiness, weakness and dizzy drowsiness had been improved.

Compared with prior art, the method for using the traditional Chinese medicine composition to prevent and treat externally diabetes in the present invention has outstanding substantive features and significant progress: 1, after having a foot bath, entrails can be restored to balance, the blood system can be restored to self-regulation under a stable blood pressure, thus decreasing the blood glucose to normal; the gastrointestinal irritation and damage on liver and kidney can be avoided as the composition is safe and healthy, heart, brain and kidney functions can also be strengthened unknowingly to avoid several complications of cardiovascular and cerebrovascular; 2, having a foot bath with the traditional Chinese composition of the present invention utilizes the principle that the boiling water can foster the medicine to filtrate into the human body, so as to ensure the medicine to penetrate the whole body meridian through the foot and adverse reactions resulted from oral overdose not to occur; the traditional Chinese medicine steam is used to fumigate the foot for more than about 10 min, then having a foot bath for 30-40 min; applying the traditional Chinese medicine fumigation into foot treatment can, by virtue of expansion of the capillaries on foot due to water steam, allow the effective ingredients of the medicine to fully pass through capillaries for circulation to body meridian and transportation to internal organs via meridian, thereby achieving the external treatment of internal disease and lower treatment of upper disease (nei bing wai zhi, shang bing xia zhi); 3, in general, during one course of having a foot bath with the medicine, the symptom of “polydipsia, polyuria, polyphagia (sanduo)” is felt to disappear, FPG23 7.8 mmol/L; having efficacy of lowering blood glucose level, enforcing spleen and consolidating kidney, harmonizing the five internal organs (heart, liver, spleen, lungs and kidneys), activating blood and dredging collaterals and dispelling wind and relieving pain, of preventing and treating such phenomenon as diabetic peripheral neuropathy, diabetic foot, diabetic nephropathy and harmfulness on human life from uremia caused by insufficient function of kidney due to diabetes It has a significant effect on preventing and treating headache, feebleness, numbness of hands and feet and complications resulted from diabetes.

### DETAILED DESCRIPTION

To better understand and implement, the following will describe the present invention with embodiments. The listed embodiments are only used to illustrate the present invention, but not for the limitation of the scope of the present invention.

Embodiment 1: weighing accurately 40 g astragalus membranaceus, 38 g salvia miltiorrhiza, 55 g stigma maydis, 45 g cortex lycii radicis, 28 g wolfberry, 28 g prunella vulgaris, 28 g mulberry leaf, 28 g cornus officinalis, 38 g lycopodium clavatum, 33 g the root of bidentate achyranthes, 45 g rhizoma chuanxiong, 28 g Chinese atractylodes, 28 g yam, 28 g puerarin, 28 g fructus arctii, 28 g cassia twig; grinding the mixture of above traditional Chinese medicines into 160-200 mesh sieve powder, then putting them into several small bags made of nonwovens respectively, each being 40 g, and enclosing as backups; putting a 40 g medicine bag into a foot pot, brewing with 2000-3000 ml of boiling water, and then steaming about 10 minutes with the traditional Chinese medicine steam after stirring evenly; when the water is cooled to 40-42° C., soaking your feet for 30-40 minutes; soaking feet once a day before going to bed and the optimum time for foot bath is about 9:00 p.m.

Process: weighing accurately 35 g astragalus membranaceus, 32 g salvia miltiorrhiza, 40 g stigma maydis, 35 g cortex lycii radicis, 26 g wolfberry, 26 g prunella vulgaris, 26 g mulberry leaf, 26 g cornus officinalis, 36 g lycopodium clavatum, 31 g the root of bidentate achyranthes, 42 g rhizoma chuanxiong, 26 g Chinese atractylodes, 26 g yam, 26 g puerarin, 26 g fructus arctii, 26 g cassia twig; grinding the mixture of above traditional Chinese medicines into 160-200 mesh sieve powder, then putting them into several small bags made of nonwovens respectively, each being 40 g, and enclosing as backups; when in use, putting a 50 g medicine bag into a foot pot, brewing with 2000-3000 ml of boiling water, and then steaming about 10 minutes with the traditional Chinese medicine steam after stirring evenly; when the water is cooled to 40-42° C., soaking your feet for 30-40 minutes; soaking feet once a day before going to bed and the optimum time for foot bath is about 9:00 p.m.

Embodiment 3: weighing accurately 45 g astragalus membranaceus, 38 g salvia miltiorrhiza, 50 g stigma maydis, 45 g cortex lycii radicis, 29 g wolfberry, 29 g prunella vulgaris, 29 g mulberry leaf, 29 g cornus officinalis, 39 g lycopodium clavatum, 34 g the root of bidentate achyranthes, 48 g rhizoma chuanxiong, 29 g Chinese atractylodes, 29 g yam, 29 g puerarin, 29 g fructus arctii, 29 g cassia twig; making above traditional Chinese medicines into decoction, adding water to decoct the Chinese herbs with suitable equipment such as casserole, ceramics, stainless steel appliances and decocting 1-2 hours with soft fire after the medicine is boiled, and concentrating into 300 ml liquid, one medicine being able to be decocted twice, then putting them into several small bags respectively, each being 40 ml, and enclosing as backups; when in use, putting 30 ml concentrate into a foot pot, brewing with 2000-3000 ml of boiling water, and then steaming about 10 minutes with the traditional Chinese medicine steam after stirring evenly; when the water is cooled to 40-42° C., soaking your feet for 30-40 minutes; soaking feet once a day before going to bed and the optimum time for foot bath is about 9:00 p.m.

Embodiment 4: weighing accurately 50 g astragalus membranaceus, 40 g salvia miltiorrhiza, 60 g stigma maydis, 50g cortex lycii radicis, 30 g wolfberry, 30 g prunella vulgaris, 30 g mulberry leaf, 30 g cornus officinalis, 40 g lycopodium clavatum, 35 g the root of bidentate achyranthes, 50 g rhizoma chuanxiong, 30 g Chinese atractylodes, 30 g yam, 30 g puerarin, 30 g fructus arctii, 30 g cassia twig; making above traditional Chinese medicines into decoction, adding water to decoct the Chinese herbs with suitable equipment such as casserole, ceramics, stainless steel appliances and decocting 1-2 hours with soft fire after the medicine is boiled, and concentrating into 300 ml liquid, one medicine being able to be decocted twice, then putting them into several small bags respectively, each being 50 ml, and enclosing as backups; when in use, putting 50 ml concentrate into a foot pot, brewing with 2000-3000 ml of boiling water, and then steaming about 10 minutes with the traditional Chinese medicine steam after stirring evenly; when the water is cooled to 40-42° C., soaking your feet for 30-40 minutes; soaking feet once a day before going to bed and the optimum time for foot bath is about 9:00 p.m.

Embodiment 5: weighing accurately 40 g astragalus membranaceus, 35 g salvia miltiorrhiza, 50 g stigma maydis, 40 g cortex lycii radicis, 25 g wolfberry, 25 g prunella vulgaris, 25 g mulberry leaf, 25 g cornus officinalis, 35 g lycopodium clavatum, 30 g the root of bidentate achyranthes, 40 g rhizoma chuanxiong, 25 g Chinese atractylodes, 25 g yam, 25 g puerarin, 25 g fructus arctii, 25 g cassia twig; making above traditional Chinese medicines into decoction, adding water to decoct the Chinese herbs with suitable equipment such as casserole, ceramics, stainless steel appliances and decocting 1-2 hours with soft fire after the medicine is boiled, and concentrating into 300 ml liquid, one medicine being able to be decocted twice, then putting them into several small bags respectively, each being 45 ml, and enclosing as backups; when in use, putting 45 ml concentrate into a foot pot, brewing with 2000-3000 ml of boiling water, and then steaming about 10 minutes with the traditional Chinese medicine steam after stirring evenly; when the water is cooled to −2-42° C., soaking your feet for 30-40 minutes; soaking feet once a day before going to bed and the optimum time for foot bath is about 9:00 p.m.

1. A traditional Chinese medicine composition for preventing and treating externally diabetes, characterized in that, the traditional Chinese medicine composition is composed of astragalus membranaceus, salvia miltiorrhiza, stigma maydis, cortex lycii radicis, wolfberry, Prunella vulgaris, mulberry leaf, cornus officinalis, lycopodium clavatum, the root of bidentate achyranthes, rhizoma chuanxiong, Chinese atractylodes, yam, puerarin, fructus arctii, cassia twig.

2. The traditional Chinese medicine composition for preventing and treating externally diabetes according to claim 1, characterized in that, the composition comprises the following weight ratios of Chinese herbal medicine: 40-50 parts of astragalus membranaceus, 35-40 parts of salvia miltiorrhiza, 50-60 parts of stigma maydis, 40-50 parts of cortex lycii radicis, 25-30 parts of wolfberry, 25-30 parts of Prunella vulgaris, 25-30 parts of mulberry leaf, 25-30 parts of cornus officinalis, 35-40 parts of lycopodium clavatum, 30-35 parts of the root of bidentate achyranthes, 40-50 parts of rhizoma chuanxiong, 25-30 parts of Chinese atractylodes, 25-30 parts of yam, 25-30 parts of puerarin, 25-30 parts of fructus arctii, 25-30 parts of cassia twig.

3. The traditional Chinese medicine composition for preventing and treating externally diabetes according to claim 2, characterized in that, the composition comprises the following weight ratios of Chinese herbal medicine: 35-45 parts of astragalus membranaceus, 32-38 parts of salvia miltiorrhiza, 40-50 parts of stigma maydis, 35-45 parts of cortex lycii radicis, 26-29 parts of wolfberry, 26-29 parts of prunella vulgaris, 26-29 parts of mulberry leaf, 26-29 parts of cornus officinalis, 36-39 parts of lycopodium clavatum, 31-34 parts of the root of bidentate achyranthes, 42-48 parts of rhizoma chuanxiong, 26-29 parts of Chinese atractylodes, 26-29 parts of yam, 26-29 parts of puerarin, 26-29 parts of fructus arctii, 26-29 parts of cassia twig.

4. The traditional Chinese medicine composition for preventing and treating externally diabetes according to claim 3, characterized in that, the composition comprises the following weight ratios of Chinese herbal medicine: 45 parts of astragalus membranaceus, 38 parts of salvia miltiorrhiza, 55 parts of stigma maydis, 45 parts of cortex lycii radicis, 28 parts of wolfberry, 28 parts of Prunella vulgaris, 28 parts of mulberry leaf, 28 parts of cornus officinalis, 38 parts of lycopodium clavatum, 33 parts of the root of bidentate achyranthes, 45 parts of rhizoma chuanxiong, 28 parts of Chinese atractylodes, 28 parts of yam, 28 parts of puerarin, 28 parts of fructus arctii, 28 parts of cassia twig.

5. The traditional Chinese medicine composition for preventing and treating externally diabetes according to claim 1, characterized in that, the Chinese medicine composition is a powder or a decoction.

6. The traditional Chinese medicine composition for preventing and treating externally diabetes according to claim 2, characterized in that, the Chinese medicine composition is a powder or a decoction.

7. The traditional Chinese medicine composition for preventing and treating externally diabetes according to claim 3 characterized in that, the Chinese medicine composition is a powder or a decoction.

8. The traditional Chinese medicine composition for preventing and treating externally diabetes according to claim 4, characterized in that, the Chinese medicine composition is a powder or a decoction.

9. The method for using the traditional Chinese medicine composition for preventing and treating externally diabetes according to claim 1, characterized in that, it comprises the following steps: putting a 40-50 g/ml medicine bag of Chinese herbal medicine powder or decoction into a foot pot, brewing with 2000-3000 ml of boiling water, and then steaming more than 10 minutes with the traditional Chinese medicine steam after stirring evenly; when the water is cooled to 40-42° C., soaking your feet for 30-40 minutes; soaking feet once a day before going to bed and a course for foot soaking is 20 days.

10. The method for using the traditional Chinese medicine composition for preventing and treating externally diabetes according to claim 2, characterized in that, it comprises the following steps: putting a 40-50 g/ml medicine bag of Chinese herbal medicine powder or decoction into a foot pot, brewing with 2000-3000 ml of boiling water, and then steaming more than 10 minutes with the traditional Chinese medicine steam after stirring evenly; when the water is cooled to 40-42° C., soaking your feet for 30-40 minutes; soaking feet once a day before going to bed and a course for foot soaking is 20 days.

11. The method for using the traditional Chinese medicine composition for preventing and treating externally diabetes according to claim 3, characterized in that, it comprises the following steps: putting a 40-50 g/ml medicine bag of Chinese herbal medicine powder or decoction into a foot pot, brewing with 2000-3000 ml of boiling water, and then steaming more than 10 minutes with the traditional Chinese medicine steam after stirring evenly; when the water is cooled to 40-42° C., soaking your feet for 30-40 minutes; soaking feet once a day before going to bed and a course for foot soaking is 20 days.

12. The method for using the traditional Chinese medicine composition for preventing and treating externally diabetes according to claim 4, characterized in that, it comprises the following steps: putting a 40-50 g/ml medicine bag of Chinese herbal medicine powder or decoction into a foot pot, brewing with 2000-3000 ml of boiling water, and then steaming more than 10 minutes with the traditional Chinese medicine steam after stirring evenly; when the water is cooled to 40-42° C., soaking your feet for 30-40 minutes; soaking feet once a day before going to bed and a course for foot soaking is 20 days.

13. The method for using the traditional Chinese medicine composition for preventing and treating externally diabetes according to claim 5, characterized in that, it comprises the following steps: putting a 40-50 g/ml medicine bag of Chinese herbal medicine powder or decoction into a foot pot, brewing with 2000-3000 ml of boiling water, and then steaming more than 10 minutes with the traditional Chinese medicine steam after stirring evenly; when the water is cooled to 40-42° C., soaking your feet for 30-40 minutes; soaking feet once a day before going to bed and a course for foot soaking is 20 days.
